(19) United States
(12) Patent Application Publication
(10) Pub. No.: US 2026/0137764 A1
(43) Pub. Date: May 21, 2026

(54) METHOD FOR CASTRATION OF ANIMALS

(71) Applicant: SHENZHEN HERZ LIFE SCIENCE TECHNOLOGY CO., LTD, Shenzhen, Guangdong (CN)

(72) Inventors: Lisha ZHA, Shenzhen (CN); Yuhang ZHOU, Shenzhen (CN); Qi ZHENG, Shenzhen (CN); Maobai WU, Shenzhen (CN)

(73) Assignee: SHENZHEN HERZ LIFE SCIENCE TECHNOLOGY CO., LTD, Shenzhen, Guangdong (CN)

(21) Appl. No.: 18/699,977

(22) PCT Filed: Dec. 7, 2023
(86) PCT No.: PCT/CN2023/136969

§ 371 Date: Apr. 10, 2024

(51) Int. Cl. A61K 39/00; A61D 7/00; A61P 37/04

(52) U.S. Cl. CPC A61K 39/0006; A61D 7/00; A61P 37/04; A61K 2039/5258; A61K 2039/54; A61K 2039/552; A61K 2039/575

(57) ABSTRACT

A method for castration of animals. A non-surgical method for castration of animals which includes administering GnRH-I-AP205 virus-like particle subunit vaccine to animals. The required dose is very small, and 0.25 ml/animal will work.

### FIELD OF THE INVENTION

The invention relates to the field of biotechnology, in particular to a method for castration of animals.

### BACKGROUND OF THE INVENTION

Castration involves externally removing the reproductive system or make the animal lose its sexual function. Castration is a common operation in animal husbandry, which can be carried out for animals of different age groups. The purpose of castration is to reduce the feed loss caused by sexual activity in the fattening stage of animals, reduce the injury and death caused by estrus and aggressive behavior of male animals, improve the quality of meat (such as reducing the smell and odor), and make use of the growth characteristics of castrated animals to gain weight. Therefore, castration is an important technical link in livestock fattening and production.

Methods of castration include chemical methods, medical castration or surgery. Among them, surgery is the most commonly used castration method, including surgical removal of testicles, ovaries or gonads. Chemical methods include the use of drugs to inhibit the secretion of sex hormones. Medical castration involves inhibiting the production of androgens in the testis by injecting drugs, such as “luteinizing hormone-releasing hormone analog”.

For centuries, except for male animals for breeding, most male animals have been castrated by surgery, which is very stressful to animals. It usually takes 7-10 days to recover, and sometimes animals die because of wound infection. In addition, the surgical castration of female animals not only has a high risk of infection, but also requires high skills and physical strength of operators. In addition, in many countries and regions, activists in animal welfare have been exerting pressure to stop surgical castration. Therefore, there is a great need for a cheap and effective method to castrate animals in a safe and harmless way in the fields of food animal production and pets.

### SUMMARY OF THE INVENTION

In view of this, the technical problem to be solved by the invention is to provide a non-surgical method for castration of animals.

The method for castration of animals described in the invention includes administering GnRH-I-AP205 virus-like particle subunit vaccine.

In the embodiment of the invention, the mode of administration is injection.

In some embodiments, the injection is intramuscular injection, intravenous injection, subcutaneous injection or intradermal injection.

In some specific embodiments, the location of the subcutaneous injection is the region between the scapulae behind the neck, the back, the ear root, the abdomen, the lateral leg or the medial leg. Preferably, the subcutaneous injection described in the method of the invention is subcutaneous injection at the neck behind the ear.

In the embodiment of the invention, the dose of the injection is 0.25-1.0 mL/animal. Preferably, the dose of the injection is 0.25-0.5 mL/animal, or 5-1.0 mL/animal. More preferably, the dose of the injection is 0.25 ml/animal, 0.5 ml/animal and 1.0 ml/animal.

In some embodiments, the injection is carried out for 1 to 3 times, for example, 1, 2 or 3 times. Preferably, the number of injections is 2. The interval between two injections is 3-5 weeks. As a feasible case, the interval between the two injections is 3 weeks, 4 weeks or 5 weeks.

Preferably, the procedure of the injection is to inject 0.25-1.0 mL/animal for the first time, and again 0.25-1.0 mL/animal after 4 weeks.

The doses of the two injections can be equal or different, which is not limited in the invention. For example, the doses of the first injection and the second injection are independently selected from 0.25 ml/animal, 0.5 ml/animal and 1.0 ml/animal. In a specific embodiment, the dose of the first injection is equal to that of the second injection. Preferably, the doses of the two injections are both 0.5 ml/animal.

In the invention, the time for the castration to generate immunity is 5 weeks after the first injection and the duration of immunity is 12 months.

In the invention, the animal is a mammal. For example, the animal is a feline, a canine or a rodent.

Based on safety considerations, the animal is a non-pregnant animal.

In the invention, the feline is a lion, a tiger, a leopard or a cat; the canid includes dogs, foxes, hyenas, wolves or jackals; and the rodent includes rats, squirrels or rabbits.

In some embodiments, the animal is a cat, and the cat is 4 -24 months old. For example, the cat is 4-12 months old, or 12-18 months old, or 18-24 months old. Specifically, the cat is 4 months old, 5 months old, 6 months old, 7 months old, 8 months old, 9 months old, 10 months old, 11 months old, 12 months old, 13 months old, 14 months old, 15 months old, 16 months old, 17 months old, 18 months old, 19 months old, 20 months old, 21 months old, 22 months old, 23 months old or 24 months old.

The invention does not limit the weight of animals, and animals in the reproductive period are all within the scope of the implementation of the solution of the invention. In some embodiments, the weight of the cat is 2-6 kg. In the embodiments of the invention, for the dose of the vaccine, the injection dose is calculated according to the animal weight of 2-6 kg. If the animal's weight is less than 2 kg or more than 6 kg, the animal's weight is converted in equal proportion according to the relationship between the animal's weight and dose described in the embodiment.

In the invention, the content of aluminum hydroxide adjuvant in the GnRH-I-AP205 virus-like particle subunit vaccine is 400 μg/ml. The mass ratio of antigen (GnRH-I-AP205) to adjuvant is 1:(0.1-10). Preferably, the mass ratio of antigen to adjuvant in the subunit vaccine is 1:0.1, 1:0.5, 1:1, 1:2, 1:5 or 1:10.

Further, the invention provides use of the aforementioned method in castration of male animals.

In some embodiments, the castration includes: increasing GnRH protein antibody titer, reducing testosterone level, reducing testicular volume or quality, reducing sperm viability and/or inhibiting mounting behavior.

Furthermore, the invention provides use of the aforementioned method in castration of female animals.

In some embodiments, the castration includes increasing GnRH protein antibody titer, reducing estradiol level and/or avoiding pregnancy.

The invention provides a non-surgical method for castration of animals, which comprises administering GnRH-I-AP205 virus-like particle subunit vaccine to animals. This method has good safety, is free of irritation or allergic reaction, and it is still safe at overdose. Also, the required dose is very small, and 0.25 ml/animal can work.

### BRIEF DESCRIPTION OF THE DRAWINGS

FIG. 1 shows the serum GnRH antibody levels in each group;

FIG. 2 shows the serum testosterone levels in each group;

FIG. 3 shows the serum estradiol levels in each group; and

FIG. 4 shows the changes of serum GnRH antibody levels with time in each group.

### DETAILED DESCRIPTION OF THE INVENTION

The invention provides a method for castration of animals, and those skilled in the art can learn from the content provided herein to appropriately improve the realization of technological parameters. It is particularly important to point out that all similar replacements and modifications are obvious to those skilled in the art, and they are all considered to be included in the invention. The method and application of the invention have been described through preferred embodiments, and obviously, modifications or appropriate changes and combinations can be made to the method and application herein without departing from the content, spirit and scope of the invention, so as to realize and apply the technology of the invention.

Unless otherwise defined in the invention, the scientific and technical terms related to the invention shall have the meanings understood by those of ordinary skill in the art.

“And/or” describes the relationship of related objects, indicating that there can be three kinds of relationships, for example, A and/or B, which can indicate that A exists alone, A and B exist at the same time, and B exists alone, wherein A and B can be singular or plural.

The terms “including”, “comprising” and “having” are used interchangeably, which are intended to indicate the inclusion of a solution, meaning that the solution may have other elements besides the listed elements. At the same time, it should be understood that the descriptions of “including”, “comprising” and “having” used herein also provide the solution of “consisting of”. “Injection” refers to the process and method of injecting liquid or gas into the body by using medical devices such as syringes, steel needles, indwelling needles and catheters to achieve the purpose of diagnosis and treatment.

The samples used in the invention are all common commercial products, which can be purchased in the market. In the examples, the subjects participating in the experiment are recorded as “preparations” in some examples, which are virus-like particle subunit vaccines of GnRH. Specifically, its vector is AP205 phage capsid protein. The preparation method thereof refers to Chinese Patent Application No. 202011399282.9.

The invention will be further illustrated with Examples.

### Example 1Minimum Inoculation Dose Test

Grouping: 30 healthy cats (15 females and 15 males) were divided into 5 groups, with 3 females and 3 males in each group, of which 1 group was the control group and the other 4 groups were the injection groups.

Inoculation: A batch of preparations were injected subcutaneously into cats at the doses of 0.13 ml/cat, 0.25 ml/cat, 0.5 ml/cat and 1.0 ml/cat at the neck behind the ear, and a group of test cats received injection in each dose group, and the second injection was made at the same dose by the same route after 28 days. The control group was fed and observed under the same conditions without injection.

GnRH protein antibody titer: on the 14th day after the second injection, the serum GnRH protein antibody titers of the cats in the control group were all <1:300; the serum GnRH protein antibody titers of the cats in the dose group of 0.13 ml/cat were all ≤1:600; the serum GnRH protein antibody titers in the dose group of 0.25 ml/cat were between 1:900 and 1:1500, and the serum GnRH protein antibody titers of the cats in the dose groups of 0.5 ml/cat and 1.0 ml/cat were all ≥1:1500.

Testicular volume of male cats: The testicular volume of male test cats in both the dose group of 0.13 ml/cat and the control group increased, the testicular volume of three male test cats in the dose group of 0.25 ml/cat decreased, and the testicular volume of all male test cats in the dose group of 0.5 ml/cat and the dose group of 1.0 ml/cat decreased.

### Test in the Same Cage

Mounting behavior of male cats: The male test cats in the control group and the dose group of 0.13 ml/cat all showed obvious mounting behavior, while all the male test cats in the dose group of 0.25 ml/cat, the dose group of 0.5 ml/cat and the dose group of 1.0 ml/cat did not show mounting behavior.

Pregnancy of female cats: the 3 female test cats and the female cats paired with the 3 male test cats in the control group in the same cage were pregnant, and 6/6 cats were in estrus; All three female test cats in the dose group of 0.13 ml/cat were pregnant, and all the female cats paired with the three male test cats in the same cage were pregnant, and 6/6 were in estrus. None of the three female test cats in the dose group of 0.25 ml/cat was pregnant, none of the female cats paired with the three male test cats in the same cage was pregnant, and 6/6 were protected; in the dose groups of 0.5 ml/cat and 1.0 ml/cat, the three female test cats and the female cats paired with the three male test cats in the same cage were not pregnant, and 6/6 were protected.

### Example 2 Inoculation Procedure Determination Test

Totally, 96 cats were tested. The tested cats received two injections and three injections respectively. The two-injection group and three-injection group, with 12 mice in each group, were vaccinated with the vaccine, the 12 mice in the control group were vaccinated with normal saline, and the other 60 mice were used for breeding. The results show that:
the serum GnRH protein antibody titers of the cats in the control group were all <1:300, and the testicular volume of six male test cats increased, and all six male test cats showed mounting behavior after being paired with female cats in the same cage;
the serum GnRH protein antibody titers of the cats in the two-injection group ranged from 1:600 to 1:900, and the testicular volume of four male test cats increased, and four male test cats showed mounting behavior after being paired with female cats in the same cage;
the serum GnRH protein antibody titers of the cats in the three-injection group were all ≥1:1500, and the testicular volume of 6 male test cats was reduced, and 6 male test cats did not show mounting behavior after being paired with female cats in the same cage.

On the 30th day after the cage test, all six female test cats in the control group were pregnant, and 6/6 cats were in estrus, while all the female cats paired with the six male test cats of the control group in the same cage were pregnant, and 6/6 cats were in estrus. Four female test cats in the two-injection group were pregnant, and 2/6 cats were protected. Female cats paired with four male test cats of the two-injection group in the same cage were pregnant, and 2/6 cats were protected. All the six female test cats in the three-injection group were not pregnant, and 6/6 cats were protected. Female cats paired with the six male test cats of the two-injection group in the same cage were not pregnant, and 6/6 cats were protected.

According to the above test results, the vaccination procedure is determined as follows: healthy cats aged 4-24 months received injection for the first time, received the second injection after 28 days and the third injection after 56 days, 0.5 ml each time.

### Example 3 Efficacy Test

A total of 128 cats were tested. Three batches of preparations were inoculated into cats, and the second injection was carried out at the same dose by the same route after 28 days. The results show that:

### 1. Antibody Titer

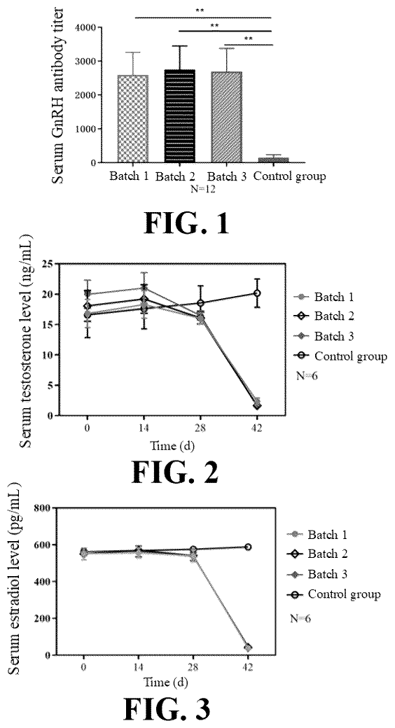

On the 14th day after the second injection, the serum GnRH protein antibody titers of the cats in the groups injected with the three batches of preparations were all ≥1:1500, and the serum GnRH protein antibody titers of the cats in the control group were all <1:300. There were significant differences in antibody between each of the groups injected with the three batches of preparations and the control group (all P<0.01) (FIG. 1);

### 2. Testicular Volume

On the 42nd and 56th day after the first injection, the testicular volume of the male cats in the groups injected with the three batches of preparations decreased, while that of the male cats in the control group increased, and there was no significant difference in testicular volume between each of the groups injected with the three batches of preparations and the control group (all P>0.05);
On the 70th day after the first injection, the testicular volume of the male cats in the groups injected with the three batches of preparations decreased, while that of the male cats in the control group increased. There were significant differences in testicular volume between each of the groups injected with the three batches of preparations and the control group (all P<0.05);

### 3. Testosterone Level

On the 42nd day after the first injection, compared with the testosterone levels of the male cats in the control group, the male cat's testosterone levels in the groups injected with the three batches of preparations all decreased, which were all lower than 5 ng/ml, and the male cat's testosterone levels between each of the groups injected with the three batches of preparations and the control group were significantly different (all P<0.01);

The changes are as shown in FIG. 2: At the 28th day after the first injection, the serum testosterone concentration of male cats in each group (one batch of preparations were injected in each group) was significantly lower than that in the control group (P<0.05). On the 42th day after the first injection, the serum testosterone concentration was significantly lower than that of the control group (P <-0.01).

### 4. Estradiol Level

On the 42nd day after the first injection, compared with the estradiol levels of the female cats in the control group, the female cat's estradiol levels in the groups injected with the three batches of preparations were all significantly reduced, which were all lower than 100 pg/ml. There were significant differences in the female cat's estradiol levels between each of the groups injected with the three batches of preparations and the control group (all P<0.01).

The changes are as shown in FIG. 3. Serum estradiol concentration: On the 28th day after the first injection, the serum estradiol concentration of female cats in each group (one batch of preparations were injected in each group) was significantly lower than that in the control group (P <−0.05). On the 42th day after the first injection, the serum estradiol concentration was significantly lower than that of the control group (P<0.01).

### 5. Sperm Viability

On the 42th day after the first injection, the sperm viability of male cats in the groups injected with the three batches of preparations decreased, while that of male cats in the control group was normal.

### 6. Test in the Same Cage

On the 14th day after the second injection, the test in the same cage was conducted, and observation was carried out for 21 consecutive days. All the male test cats in the groups injected with the three batches of preparations did not appear mounting behavior and were not in estrus, and 6/6 cats were protected. In the control group, all the six male test cats showed multiple mounting behaviors, and 6/6 cats were in estrus. On the 30th day after the test in the same cage, none of the female cats paired with the male test cats in the groups injected with the three batches of preparations was pregnant, and 6/6 cats were protected, none of the female test cats in the groups injected with the three batches of preparations was pregnant, and 6/6 cats were protected. Six female test cats in the control group were all pregnant, and 6/6 cats were in estrus; in the control group, the female cats paired with the 6 male test cats were pregnant, and 6/6 cats were in estrus.

The above results show that the preparation was inoculated to healthy cats aged 4-24 months at a dose of 0.5 ml/cat, and then injected for the second time at the same dose by the same route 28 days later, which made the cats produce GnRH protein antibodies. On the 14th day after the second injection, the serum GnRH protein antibody titers of the cats in the injection group were ≥1:1500, which inhibited the cat's reproductive ability.

### Example 4 Efficacy Duration and Antibody Dynamic Rule Test

A total of 64 cats were tested, and three batches of preparations were inoculated into cats. After 28 days, the second injection was carried out at the same dose by the same route, while the control group did not receive injection.

The results show that the serum GnRH protein antibody titers of the cats can reach 1:900-1:1500 at 5 weeks after the first injection, and reached the peak at 7-8 weeks after injection. The serum GnRH protein antibody titers of the cats in the injection groups were between 1:1500 and 1:3900, and then the GnRH protein antibody level began to decrease gradually, until 13 months after injection, the serum GnRH protein antibody level of the cats in the injection groups was still not lower than 1:1500, while the serum GnRH protein antibody level of the cats in the control group was less than 1:300. Until 13 months in the test in the same cage, 6/6 cats in the injection groups were protected and 6/6 cats in the control group were in estrus.

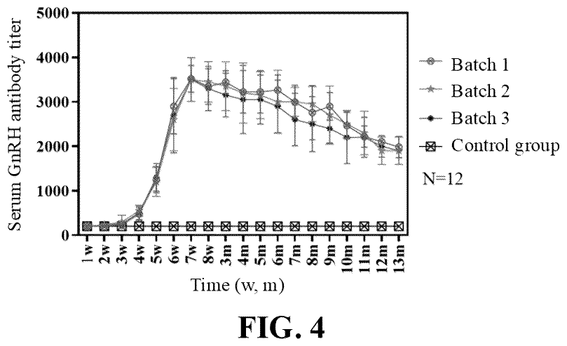

According to the results of the GnRH antibody test and the test in the same cage, in the case that the preparation was inoculated to healthy cats aged 4-24 months, at 0.5 ml/cat, and the second injection was carried out with the same dose and route after 28 days, the time to generate immunity was 5 weeks after the first injection and the immunity duration was 13 months (FIG. 4).

### Example 5 Safety Study

### 1. Safety Test of Mice Inoculated With Overdose

Test animals: 40 mice, 10 mice in each group, 3 test groups and 1 control group.

The results show that three batches of preparations were inoculated to 6-week-old healthy Kunming mice in overdose (600 μg/mouse). After continuous observation for 14 days after injection, there were no obvious changes in drinking water, food intake and spirit of the mice, no systemic adverse reactions, no local adverse reactions such as redness and lumps at the injection site, and no pathological changes at the injection site, liver and kidney at autopsy. There were no obvious differences from the control group. There were no pathological changes at the injection site, liver and kidney after autopsy.

### 2. Local Irritation Test

Test animals: 10 cats, 5 in each group, 1 test group and 1 control group.

The results show that one batch of preparations was injected with a single dose (200 μg/cat), and there was no obvious inflammation, swelling, lumps and other local adverse reactions at the injection site. Local tissues were removed for microscopic examination, and there was no pathological change at the injection site.

### 3. Systemic Anaphylaxis Test

Test animals: 20 cats, 5 in each group, 3 test groups and 1 control group.

The results show that three batches of preparations were inoculated (200 μg/cat) for the second time after 28 days, and continuous observation was carried out for 14 days. The cats in each injection group did not have allergic symptoms such as systemic shock, skin allergy, respiratory tract abnormality and digestive tract abnormality, and there was no significant difference from the control group.

4. Single Dose (200 μg/Animal) Safety Test

Test animals: 10 cats, 5 in each group, 1 test group and 1 control group.

The results show that the body temperature, food intake, drinking water and spirit of the test cats inoculated with a single dose of one batch of preparations were not significantly changed, there were no systemic adverse reactions, and there were no local adverse reactions such as redness and lumps at the injection site.

5. Single Dose (200 μg/Animal) Duplicate Safety

Test animals: 10 cats, 5 in each group, 1 test group and 1 control group.

The results show that the second inoculation was carried out 28 days after the inoculation with one batch of preparations. The temperature, food intake, drinking water, and spirit of the test cats were not significantly changed, there were no local adverse reactions such as redness, swelling and lumps at the injection site, and there were no pathological changes in all organs after autopsy.

6. Overdose (600 μg/Animal) Safety Test

Test animals: 20 cats, 5 in each group, 3 test groups and 1 control group.

The results show that after overdose inoculation with 3 batches of preparations, continuous observation was carried out for 14 days. The temperature, food intake, drinking water and spirit of the test cats had no obvious changes, there were no systemic adverse reactions, no local adverse reactions such as redness and lumps at the injection site, and no pathological changes at the injection site and in all organs after autopsy.

### 7. Safety Test on Cats of Non-Use Days Old

Test animals: 20 cats, 5 in each group, 3 test groups and 1 control group.

The results show that three batches of preparations were inoculated to healthy cats aged 2-3 months with an overdose (600 μg/cat), and continuous observation was carried out for 14 days. The body temperature, food intake, drinking water and spirit of the test cats had no obvious changes, there were no systemic adverse reactions, no local adverse reactions such as redness and lumps at the injection site, and no pathological changes at the injection site and in all organs.

### 8. Safety Test on Pregnant Female Cats

Test animals: 10 cats, 5 in each group, 1 test group and 1 control group.

The results show that one batch of preparations were inoculated at an overdose (600 μg/cat) to healthy cats female that had been pregnant for 3-6 weeks, and continuous observation was carried out for 14 days. The body temperature, food intake, drinking water and spirit of the test cats had no obvious changes, there were no systemic adverse reactions, and no local adverse reactions such as redness, swelling and lumps at the injection site. The female cats in the injection group had stillbirth, while the female cats in the control group had no abortion, stillbirth and teratogenesis. The above results show that this preparation may lead to miscarriage and stillbirth when inoculated to pregnant female cats. Therefore, this preparation is prohibited for pregnant female cats.

The above are only the preferred embodiments of the invention, and it should be pointed out that for those skilled in the art, a number of improvements and embellishments can be made without departing from the principle of the invention, and these improvements and embellishments should also be regarded as the protection scope of the invention.

1-17. (canceled)

18. A method for castration of animals, comprising administering GnRH-I-AP205 virus-like particle subunit vaccine.

19. The method according to claim 18, wherein the mode of administration is injection.

20. The method according to claim 19, wherein the injection is intramuscular injection, intravenous injection, subcutaneous injection or intradermal injection.

21. The method according to claim 20, wherein the subcutaneous injection is subcutaneous injection at the neck behind the ear.

22. The method according to claim 18, wherein the dose of the injection is 0.25-1.0 mL/animal.

23. The method according to claim 22, wherein the procedure of the injection is to inject 0.25-1.0 mL/animal for the first time, and inject 0.25-1.0 mL/animal again after 4 weeks.

24. The method according to claim 23, wherein the time for the castration to generate immunity is 5 weeks after the first injection and the duration of immunity is 12 months.

25. The method according to claim 18, wherein the animal is a feline, a canine or a rodent.

26. The method according to claim 25, wherein the animal is a non-pregnant animal.

27. The method according to claim 25, wherein the animal is a cat, a dog or a mouse.

28. The method according to claim 27, wherein the cat is 4 -24 months old.

29. The method according to claim 28, wherein the weight of the cat is 2-6 kg.

30. The method according to claim 18, wherein the content of aluminum hydroxide adjuvant in the GnRH-I-AP205 virus-like particle subunit vaccine is 400 μg/ml, and the mass ratio of GnRH-I-AP205 to adjuvant is 1:(0.1-10).

31. Use of the method according to claim 18 in castration of male animals.

32. The use according to claim 31, wherein the castration comprises: increasing GnRH protein antibody titer, reducing testosterone level, reducing testicular volume or weight, reducing sperm viability and/or inhibiting mounting behavior.

33. Use of the method according to claim 18 in castration of female animals.

34. The use according to claim 31, wherein the castration comprises: increasing GnRH protein antibody titer, reducing estradiol level and/or avoiding pregnancy.
